(19) United States
(12) Patent Application Publication
(10) Pub. No.: US 2022/0331212 A1
(43) Pub. Date: Oct. 20, 2022

(54) NON-FLAMMABLE, DRY PERSONAL CARE PRODUCT CONTAINING trans-1233zd AND ETHANOL

(71) Applicant: Honeywell International Inc., Morris Plains, NJ (US)

(72) Inventors: Anthony ANZALONE, Morris Plains, NJ (US); Chantelle MOORE, Morris Plains, NJ (US); Leonard Michael STACHURA, Morris Plains, NJ (US); Yiu Keung LING, Morris Plains, NJ (US)

(21) Appl. No.: 17/753,283

(22) PCT Filed: Aug. 28, 2020
(86) PCT No.: PCT/US2020/048597

§ 371 Date: Feb. 25, 2022

Related U.S. Application Data
(60) Provisional application No. 62/892,947, filed on Aug. 28, 2019.

(51) Int. Cl. A61K 8/34; A61K 8/31; A61K 8/73; A61K 8/25; A61Q 5/00; A45D 19/00; A45D 34/04

(52) U.S. Cl. CPC A61K 8/34; A61K 8/31; A61K 8/732; A61K 8/25; A61Q 5/00; A45D 19/00; A45D 34/04

(57) ABSTRACT

Disclosed are devices and methods for applying a hair and/or skin and/or scalp care product using a a personal care formulation having an active component for treating the hair and/or scalp and/or skin of the user and a carrier for the active component, wherein the carrier comprises from about 4% to about 10% by weight based on the contents of the container of trans-1233zd and from about 1% to less than 5% by weight ethanol, where in the weight ratio of said trans-1233zd:ethanol is at least about 2.

### CROSS REFERENCE TO RELATED APPLICATIONS

This application claims the priority benefit to U.S. Provisional application No. 62/892,947, filed Aug. 28, 2019, which is hereby incorporated by reference in its entirety.

### BACKGROUND

Many personal care products are applied in the form of a spray, including hair sprays, dry shampoo, deodorants, and antiperspirants. Such products generally comprise an active component or components that provide the intended function when applied to the skin, hair or scalp. In many important applications, the intended function is remove oil from the surface being treated. Such products also generally include carriers which solvate, disperse and/or emulsify the active ingredient so that it can be effectively transported from its container to the surface of the user to be treated. In many applications, the container includes a propellant or other means to propel the active/carrier to the surface to be treated in the form of a spray. As a result of the application of the composition in the intended manner, the active component is able to provide its intended benefit and the carrier leaves the surface, usually by evaporation of the carrier.

Generally, it is desired for personal care products of the type according to the present invention to deliver the active ingredient in the form of a well-formed spray that dries quickly from the skin, hair and/or scalp of the user. It has also become increasing desirable for the carrier, and the propellant when used, to have a relatively small negative environmental impact, such as having a low ozone depletion potential, a low global warming potential and low volatile organics content. It is also highly desirable for many applications that the formulation is non-flammable.

One carrier that has heretofor been commonly used in such applications is ethanol. Applicants have come to appreciate that important and unexpected improvements to such products can be achieved using novel carriers, including novel combinations of carrier and propellant, in a spray-applied personal care formulation that can provide a well-formed spray that is non-flammable, has a low environmental impact and which can be ejected reliable from the spray nozzle with repeated use.

### SUMMARY

Applicants have found that the preferred personal care compositions, products and methods of the present invention, especially and preferably when the active component comprises an oil absorbing component, have the unexpected advantage of being non-flammable and yet reliably sprayable over numerous sprayings by using as carrier for the active component of the personal care product a combination of ethanol and trans-1-chloro,3,3,3-trifluoropropene (trans1233zd), where the amounts of ethanol and trans1233zd present in the carrier are as described herein.

Applicants have unexpectedly found that operating with amounts of ethanol below the preferred minimum amounts described herein causes significant problems with the ability of the personal care product to be released reliably and repeatedly from the container, especially and preferably when the active component comprises an oil absorbing component, and that operating with amounts of ethanol greater than the preferred maximum amounts herein tends to cause the material that is released to be flammable.

In preferred embodiments the active component of the personal care product comprises, consists essentially of or consist of an oil absorbing component. As the term is used herein, the term “oil absorbing component” means a component or combination of components that function to tend to remove one or more of the oils that are present on or in the skin, scalp or hair being treated.

In preferred embodiments the personal care product comprises an oil absorbing component that comprises, consists essentially of, or consists of one or more of: personal care starch; personal care cleansing polymer; personal care silica; personal care clay; and combinations of these. As the terms are used herein, “personal care starch,” “personal care cleansing polymer,” “personal care silica,” “personal care clay,” mean the respective component in a form that is understood by those skilled in the art to be suitable and effective for use in personal care spray formulations.

### BRIEF DESCRIPTION OF THE DRAWING

FIG. 1 is an illustration of an exemplary container containing the personal care formulation of the present invention.

### DETAILED DESCRIPTION

### Active Components

As described above, the personal care compositions of the present invention comprise an active component. While it is contemplated that broadly speaking the present invention relates to a wide variety of active components, in preferred embodiments the active component comprises, consists essentially of, or consists of a personal care oil absorbing component which has as its function the removal of oil(s) from the skin, scalp and/or hair of the user. In some preferred embodiments, as described for example more fully hereinafter, the oil absorbing component may also have one or more additional functions that are desirable or beneficial in connection to the user.

Generally speaking, the amount of the active component, and the oil absorbing component in particular, can vary widely based upon the particular application involved. In preferred embodiments, the personal care compositions of the present invention comprise from about 3% to about 15% by weight of oil absorbing component, more preferably from about 3% to about 10% by weight of oil absorbing component, and even more preferably from about 3% to about 8% of oil absorbing component.

### Starch

As mentioned above, the personal care starch can be any personal care starch known to those skilled in the art. In preferred embodiments, the personal care starch comprises, consists essentially of or consist of one or more of: rice starch, tapioca starch, corn starch, potato starch, aluminum starch and talc-starch. As the terms are used herein, each of the personal care starch components is intended to include both natural starch and starch that has been modified versions. For example, the term rice starch is intended to include not only natural rice starch but also the di-starch phospatemodified starch. As is known, starch is a natural polysaccharide consisting of amylose and amylopectin units. Modified starch may consists, for example, of natural starch molecules that have been cross linked with a phosphate group (see for example, modified rice starch (di-phosphate starch) as CAS Number 55963-33-2). An example of a potato starch for use in accordance with the present invention is available from Actera Ingredients under the trade designation TeraSorb™.

The present invention includes formulations in which the active component is a personal care starch having an average particle size of less than about 30 microns, or less than 20 microns, or less than 10 microns, with starch having an average particle size of less than about 10 microns being preferred in some formulations.

In preferred embodiments, the personal care compositions of the present invention comprise from about 1% to about 10% by weight of personal care starch, more preferably from about 2% to about 8% of personal care starch, and even more preferably from about 2% to about 6% of personal care starch.

### Polymer

The personal care polymer can be any personal care polymer known to those skilled in the art to absorb oil from skin, hair and/or scalp. In preferred embodiments, the personal care polymer comprises, consists essentially of or consist of one or more of: polyvinylpyrrolidone (PVP) and methyl methacrylate crosspolymer (PMMA). An example of a PVP personal care polymer is available from Ashland under the trade designation Advantage Revive™. In preferred embodiments, the personal care compositions of the present invention comprise from about 1% to about 10% by weight of personal care polymer, more preferably from about 1% to about 8% of personal care polymer, and even more preferably from about 1% to about 6% of personal care polymer.

### Silica

The personal care silica can be any personal care silica known to those skilled in the art to absorb oil from skin, hair and/or scalp. In preferred embodiments, the silica is a hydrated silica and/or amorphous silica and/or fumed silica. An example of a fumed personal care silica is available from Cabot Corporation under the trade designation CAB-O-SIL M5.

In preferred embodiments, the personal care compositions of the present invention comprise from about 1% to about 10% by weight of personal care silica, more preferably from about 1% to about 8% of personal care silica, and even more preferably from about 1% to about 6% of personal care silica.

### Clay

The personal care clay can be any personal care clay known to those skilled in the art to absorb oil from skin, hair and/or scalp. In preferred embodiments, the personal care clay comprises, consists essentially of or consist of one or more of Kaolin clay and/or Montmorillonite clay and/or Illite clay.

In preferred embodiments, the personal care compositions of the present invention comprise from about 1% to about 10% by weight of personal care clay, more preferably from about 1% to about 8% of personal care clay, and even more preferably from about 1% to about 6% of personal care clay.

### Delivery Device

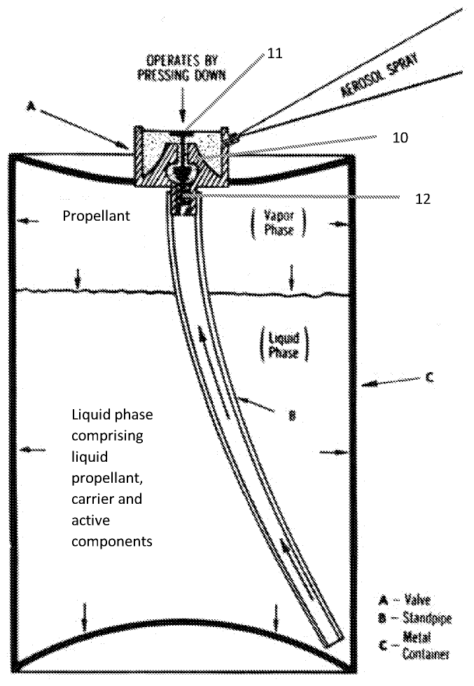

An exemplary aerosol spray device is disclosed in FIG. 1 hereof. As illustrated in the Figure, a container (typically a metal canister capable of holding fluid under pressure) has an opening closed with a spray nozzle A. The nozzle A includes a valve portion 10 and an actuator 11. A vapor space occupied by the gaseous portion of the propellant exerts a pressure on the liquid components of the personal care composition. When the actuator 11 is depressed by the user against the closing force of the spring 12, the valve is opened and the pressure exerted by the propellant forces the liquid through the dip-tube B and out of the valve in the form of an aerosol spray. When the user releases the actuator button 11, the spring reseats the valve and the flow of fluid is discontinued.

Applicants have found that preferred embodiments of the present invention have the unexpected advantage of achieving reliable operation of a personal care spray product and at the same time producing spray which is non-flammable. In particular, and without being bound by or to a particular theory of operation, applicants have found that when the carrier contains less than the preferred minimum amount ethanol as described herein, the active component of the formulation, particularly for embodiments in which the active component comprises, consists essentially of or consists of personal care starch; personal care cleansing polymer; personal care silica; personal care clay; and combinations of these, can tend to swell and/or otherwise cause the valve of the nozzle to stick at least partially in the open position. This disadvantageous result can substantially degrade the reliability of the operation of the spray device. On the other hand, applicants have found that while including amounts above the maximum preferred amounts described herein can cause the discharged spray to be flammable.

Accordingly, a preferred embodiment of the present invention provides a device for applying a hair and/or skin and/or scalp care product to a user, said device comprising:

A) a container having a spray nozzle for alternatively opening and closing the container and upon opening expelling at least a portion of contents therefrom;

B) a personal care formulation in said container comprising:

a) an active component for treating the hair and/or scalp and/or skin of the user;

b) carrier for the active component, said carrier comprising: from about 4% to about 10% by weight based on the contents of the container of trans-1233zd and

from about 1% to less than 5% by weight based on the contents of the container of of ethanol, where in the weight ratio of said trans-1233zd:ethanol is at least about 2; and

c) at least about 70% by weight based on the contents of the container of a non-flammable propellant.

It is understood that the term “propellant” as used herein (including in FIG. 1) contemplates a single component used as a propellant and blends of two or more components used as a propellant. Similarly, the terms “carrier” and “active component” as used herein contemplates a single component used as a carrier or active component and blends of two or more active components or blends of two or more components used as a carrier.

The present invention also provides a device for applying a hair and/or skin and/or scalp care product to a user, said device comprising:

A) a container having a spray nozzle for alternatively opening and closing the container and upon opening expelling at least a portion of contents therefrom;

B) a personal care formulation in said container comprising:

a) from about 1% to about 10% by weight of an active component for absorbing oil from the hair and/or scalp and/or skin of the user;

b) carrier for the active component, said carrier comprising:
i) from about 4% to about 10% by weight based on the contents of the container of trans-1233zd and
ii) from about 1% to less than 5% by weight based on the contents of the container of of ethanol, wherein the weight ratio of said trans-1233zd:ethanol is from about 2 to about 6; and

c) at least about 70% by weight based on the contents of the container of a non-flammable propellant.

The present invention also provides a device for applying a hair and/or skin and/or scalp care product to a user, said device comprising:

A) a container having a spray nozzle for alternatively opening and closing the container and upon opening expelling at least a portion of contents therefrom;

B) a personal care formulation in said container comprising:

a) from about 1% to about 10% by weight of an active component for absorbing oil from the hair and/or scalp and/or skin of the user;

b) carrier for the active component, said carrier comprising:
i) from about 4% to about 10% by weight based on the contents of the container of trans-1233zd and
ii) from about 1% to less than 5% by weight based on the contents of the container of of ethanol, wherein the weight ratio of said trans-1233zd:ethanol is greater than about 2 and wherein the weight ratio of said trans-1233zd:active component is from about 0.75:1 to about 3:1; and

c) at least about 70% by weight based on the contents of the container of a non-flammable propellant.

The present invention also provides a device for applying a hair and/or skin and/or scalp care product to a user, said device comprising:

A) a container having a spray nozzle for alternatively opening and closing the container and upon opening expelling at least a portion of contents therefrom;

B) a personal care formulation in said container comprising:

a) from about 1% to about 10% by weight of an oil absorbing personal care active component comprising one or more of personal care starch, personal care cleansing polymer, personal care silica, personal care clay, and combinations of these;

b) carrier for the active component, said carrier comprising:
i) from about 4% to about 10% by weight based on the contents of the container of trans-1233zd and
ii) from about 1% to less than 5% by weight based on the contents of the container of of ethanol, where in the weight ratio of said trans-1233zd:ethanol is at least about 2; and

c) at least about 70% by weight based on the contents of the container of a non-flammable propellant.

The present invention also provides a device for applying a hair and/or skin and/or scalp care product to a user, said device comprising:

A) a container having a spray nozzle for alternatively opening and closing the container and upon opening expelling at least a portion of contents therefrom;

B) a personal care formulation in said container comprising:

a) from about 1% to about 10% by weight of an oil absorbing personal care active component comprising one or more of personal care starch, personal care cleansing polymer, personal care silica, personal care clay, and combinations of these;

b) carrier for the active component, said carrier comprising:
i) from about 4% to about 10% by weight based on the contents of the container of trans-1233zd and
ii) from about 1% to less than 5% by weight based on the contents of the container of of ethanol, where in the weight ratio of said trans-1233zd:ethanol is from about 2 to about 6; and
c) at least about 70% by weight based on the contents of the container of a non-flammable propellant.

According to preferred embodiments as described herein, the personal care product is a dry or dry-feel personal care product spray. As those skilled in the art understand, such products are class of products that are substantially dry as the reach the user and/or are sufficiently dry to provide the used with a dry feel upon application. Such products include dry shampoo product, dry skin cleanser, dry face cleaner and the like.

### Methods

A preferred embodiment of the present invention provides methods of applying a hair and/or skin and/or scalp care product to a user comprising:

A) providing a container having a spray nozzle for expelling at least a portion of contents therefrom;

B) including in said container a personal care formulation comprising:

a) an active component for treating the hair and/or scalp and/or skin of the user; and

b) carrier for the active component, said carrier comprising:
i) from about 4% to about 10% by weight based on the contents of the container of trans-1233zd and
ii) from about 1% to less than 5% by weight based on the contents of the container of of ethanol, where in the weight ratio of said trans-1233zd:ethanol is at least about 2; and

c) at least about 70% by weight based on the contents of the container of a non-flammable propellant; and

C) spraying said formulation onto the hair and/or skin and/or scalp of the user.

It should be noted that in the methods of the present invention the concentration of the components as ejected from the container is not required to be the same as the concentration of the components within the container, although in many formulations the concentrations may be substantially the same.

The present invention also provides methods of applying a hair and/or skin and/or scalp care product to a user comprising:

A) providing a container having a spray nozzle for expelling at least a portion of contents therefrom;

B) including in said container a personal care formulation comprising:

a) from about 1% to about 10% by weight of an active component for absorbing oil from the hair and/or scalp and/or skin of the user;

b) carrier for the active component, said carrier comprising:
i) from about 4% to about 10% by weight based on the contents of the container of trans-1233zd and
ii) from about 1% to less than 5% by weight based on the contents of the container of of ethanol, where in the weight ratio of said trans-1233zd:ethanol is from about 2 to about 6; and

c) at least about 70% by weight based on the contents of the container of a non-flammable propellant; and

C) spraying said formulation onto the hair and/or skin and/or scalp of the user.

The present invention also provides methods of applying a hair and/or skin and/or scalp care product to a user comprising:

A) providing a container having a spray nozzle for expelling at least a portion of contents therefrom;

B) including in said container a personal care formulation comprising:

a) from about 1% to about 10% by weight of an oil absorbing personal care active component comprising one or more of personal care starch, personal care cleansing polymer, personal care silica, personal care clay, and combinations of these;

b) carrier for the active component, said carrier comprising:
i) from about 4% to about 10% by weight based on the contents of the container of trans-1233zd and
ii) from about 1% to less than 5% by weight based on the contents of the container of of ethanol, where in the weight ratio of said trans-1233zd:ethanol is from about 2 to about 6; and

c) at least about 70% by weight based on the contents of the container of a non-flammable propellant; and

C) spraying said formulation onto the hair and/or skin and/or scalp of the user.

### EXAMPLES

Example 1.

The formula indicated in Table 1 below was formed by mixing the indicated components, except for the propellant, and introducing them into an aerosol spray can. The aerosol cans were sealed with the valve/actuator arrangement, and then the indicated propellant was added through the valve. The formulation spray pattern was then evaluated while the nozzle is held under ambient condition (about 73F and 66 relative humidity). After the formulation passed the specific compatibility test (i.e. aerosol was actuated 2 times, 5 seconds each), then formulation was aged at room temperature for a week and checked for vapor pressure and weight loss. The designated enclosed-space test, as per the United Nations Globally Harmonized System of Classification and Labeling of Chemicals (GHS) for aerosol, which is known to those skilled in that art and available on the web at: http://www.unece.org/trans/danger/publi/ghs/ghs_rev00/00files_e.html, was conducted to evaluate whether the formulation is considered as flammable under GHS requirements.

TABLE 1

| Components | Formulation 1 |
|---|---|
| Trans-1234ze | 80 |
| Ethanol | 15 |
| Starch (prioprietary | 5 |
| personal care starch from | |
| Church and Dwight) | |

Results

| | |
|---|---|
| Formulation Compatibility | Passed |
| Test | |
| GHS Flammability | Flammable |
| Classification | |

### Example 2

Example 1 is repeated except that trans-1233zd was used as carrier, instead of ethanol. The formulations tested and the test results are reported in Table 2 below.

TABLE 2

| Components, Weight % | Formulation 2A | Formulation 2B |
|---|---|---|
| Trans-1234ze | 85 | 90 |
| Trans-1233zd | 10 | 5 |
| Ethanol | 0 | 0 |
| Starch | 5 | 5 |

Results

| | | |
|---|---|---|
| Formulation Compatibility | Failed | Failed |
| Test | | |
| GHS Flammability | Non-flammable | Non-flammable |
| Classification | | |

As can be seen from the results of the test work reported in Example 2, the formulation compatibility of formulation 2A and formulation 2B, in which the propellant consists of trans-1234ze and the carrier consists of trans-1233zd, fail because applicants have found that such formulations tend to cause the valve to stick at least partially in the open position, which in turns has a negative impact on the ability to reliably expell the formulation over repeated instances of use. However, the formulations are still classified as non-flammable according to GHS classification because there are less than 1% GHS classified flammable component in the formulation.

Example 3

Example 2 is repeated except the carrier is formulated to comprise, consist essentially of, or consists of ethanol, which is a flammable component, and trans-1233zd, according to the formulations indicated in Table 3 below:

TABLE 3

| Components, | Formulation | Formulation | Formulation | Formulation | Formulation |
|---|---|---|---|---|---|
| Weight % | 3A | 3B | 3C | 3D | 3E |
| Trans-1234ze | 80 | 85 | 85 | 90 | 90 |
| Trans-1233zd | 10 | 9 | 8 | 4 | 3.5 |
| Ethanol | 5 | 1.5 | 3 | 2 | 3 |
| Starch | 5 | 4.5 | 4 | 4 | 3.5 |

Results

| | | | | | |
|---|---|---|---|---|---|
| Trans-1233zd to | 2 | 6 | 2.67 | 2 | 1.17 |
| Ethanol Ratio | | | | | |
| Internal | Passed | Passed | Passed | Passed | Passed |
| Compatibility | | | | | |
| Test | | | | | |
| GHS | Flammable | Non- | Non- | Non- | Flammable |
| Flammability | | flammable | flammable | flammable | |
| Classification | | | | | |

The results of Example 3 demonstrate the unexpected advantage which is achieved when the carrier comprises ethanol as co-carrier with trans-1233zd. In particular, applicants have discovered that a formulation with adequate compatibility is formed by Formulation 3A and Formulation 3E, but the amount of ethanol is too high to produce an non-flammable material to be expelled. Unexpectedly, Formulation 3B, Formulation 3C and Formulation 3D are classified as non-flammable and are able to passed the GHS enclosed-space test. Accordingly, applicants have discovered a critical range of these two components to be able to at once achieve each of the beneficial properties described herein.

### Example 4

The formula indicated in Table 4 below is formed by mixing the indicated components, except for the propellant, and introducing them into an aerosol spray can. The aerosol cans are sealed with the valve/actuator arrangement, and then the indicated propellant is added through the valve. The formulation spray pattern is then evaluated while the nozzle is held under ambient condition (about 73° F. and 66 relative humidity). After the formulation is subjected to the specific compatibility test (i.e. aerosol was actuated 2 times, 5 seconds each), then the formulation is aged at room temperature for a week and checked for vapor pressure and weight loss. The designated enclosed-space test, as per GHS for aerosol, is conducted to evaluate whether the formulation is considered as flammable under GHS requirements.

TABLE 4

| Components | Formulation 4 |
|---|---|
| Trans-1234ze | 80 |
| Ethanol | 15 |
| Starch (modified rice starch | 5 |
| (di-phosphate starch) as | |
| CAS Number 55963-33-2) | |

Results

| | |
|---|---|
| Formulation Compatibility | Passed |
| Test | |
| GHS Flammability | Flammable |
| Classification | |

### Example 5

Example 4 is repeated except that trans-1233zd is used as carrier, instead of ethanol. The formulations tested and the test results are reported in Table 5 below.

TABLE 5

| Components, Weight % | Formulation 5A | Formulation 5B |
|---|---|---|
| Trans-1234ze | 85 | 90 |
| Trans-1233zd | 10 | 5 |
| Ethanol | 0 | 0 |
| Modified Rice Starch (CAS | 5 | 5 |
| Number 55963-33-2) | | |

Results

| | | |
|---|---|---|
| Formulation Compatibility | Failed | Failed |
| Test | | |
| GHS Flammability | Non-flammable | Non-flammable |
| Classification | | |

As can be seen from the results of the test work reported in Example 5, the formulation compatibility of formulation 5A and formulation 5B, in which the propellant consists of trans-1234ze and the carrier consists of trans-1233zd, fails because applicants have found that such formulations tend to cause the valve to stick at least partially in the open position, which in turns has a negative impact on the ability to reliably expell the formulation over repeated instances of use. However, the formulations are still classified as non-flammable according to GHS classification because there are less than 1% GHS classified flammable component in the formulation.

### Example 6A

Example 5 is repeated except the carrier is formulated to comprise, consist essentially of, or consists of ethanol, which is a flammable component, and trans-1233zd according to the formulations indicated in Table 6-1 below:

TABLE 6-1

| Components, | Formulation | Formulation | Formulation | Formulation | Formulation |
|---|---|---|---|---|---|
| Weight % | 6A | 6B | 6C | 6D | 6E |
| Trans-1234ze | 80 | 85 | 85 | 90 | 90 |
| Trans-1233zd | 10 | 9 | 8 | 4 | 3.5 |
| Ethanol | 5 | 1.5 | 3 | 2 | 3 |
| Modified Rice | 5 | 4.5 | 4 | 4 | 3.5 |
| Starch (CAS | | | | | |
| Number 55963- | | | | | |
| 33-2) | | | | | |

Results

| | | | | | |
|---|---|---|---|---|---|
| Trans-1233zd to | 2 | 6 | 2.67 | 2 | 1.17 |
| Ethanol Ratio | | | | | |
| Internal | Passed | Passed | Passed | Passed | Passed |
| Compatibility | | | | | |
| Test | | | | | |
| GHS | Flammability | Non- | Non- | Non- | Flammable |
| Flammable | | flammable | flammable | flammable | |
| Classification | | | | | |

The results of Example 6A demonstrate the unexpected advantage which is achieved when the carrier comprises ethanol as co-carrier with trans-1233zd. In particular, applicants have discovered that a formulation with adequate compatibility is formed by Formulation 6A and Formulation 6E, but the amount of ethanol is too high to produce an non-flammable material to be expelled. Unexpectedly, Formulation 6B, Formulation 6C and Formulation 6D are classified as non-flammable and are able to passed the GHS enclosed-space test. Accoringly, applicants have discovered a critical range of these two components to be able to at once achieve each of the beneficial properties described herein.

### Example 6B

Example 5 is repeated except the carrier is formulated to comprise, consist essentially of, or consists of ethanol, which is a flammable component, and trans-1233zd according to the formulations indicated in Table 6-2 below:

TABLE 6-2

| Components, | Formulation | Formulation |
|---|---|---|
| Weight % | 6F | 6G |
| Trans-1234ze | 77 | 82.5 |
| Trans-1233zd | 10 | 9 |
| Ethanol | 5 | 1.5 |
| Modified Rice | 8 | 8 |
| Starch (CAS | | |
| Number 55963- | | |
| 33-2) | | |

Results

| | | |
|---|---|---|
| Trans-1233zd to | 2 | 6 |
| Ethanol Ratio | | |
| Deflagration | Yes | No |
| observed | | |
| GHS | Non- | Non- |
| Flammability | flammable | flammable |
| Classification | | |

The results of this Example 6B demonstrate that even with active ingredient concentrations as high as 8%, and a correspondingly lower concentration of propellant compared to the prior 5% active component examples, and an ethanol concentration of 5% (1233zd:ethanol weight ratio of 2), a non-flammable spray as measure by GHS is produced, although signs of deflagration are observed, indicating that the concentrations for that formulation are at the edge of the flammability limit.

### Example 7.

The formula indicated in Table 7 below is formed by mixing the indicated components, except for the propellant, and introducing them into an aerosol spray can. The aerosol cans are sealed with the valve/actuator arrangement, and then the indicated propellant is added through the valve. The formulation spray pattern is then evaluated while the nozzle is held under ambient condition (about 73° F. and 66 relative humidity). After the formulation is subjected to the specific compatibility test (i.e. aerosol was actuated 2 times, 5 seconds each), then the formulation is aged at room temperature for a week and checked for vapor pressure and weight loss. The designated enclosed-space test, as per GHS for aerosol, is conducted to evaluate whether the formulation is considered as flammable under GHS requirements.

TABLE 7

| Components | Formulation 7 |
|---|---|
| Trans-1234ze | 80 |
| Ethanol | 15 |
| Potato Starch | 5 |
| (TeraSorb ™) | |

Results

| | |
|---|---|
| Formulation Compatibility | Passed |
| Test | |
| GHS Flammability | Flammable |
| Classification | |

### Example 8

Example 7 is repeated except that trans-1233zd is used as carrier, instead of ethanol. The formulations tested and the test results are reported in Table 8 below.

TABLE 8

| Components, Weight % | Formulation 8A | Formulation 8B |
|---|---|---|
| Trans-1234ze | 85 | 90 |
| Trans-1233zd | 10 | 5 |
| Ethanol | 0 | 0 |
| Potato Starch (TeraSorb ™) | 5 | 5 |

Results

| | | |
|---|---|---|
| Formulation Compatibility | Failed | Failed |
| Test | | |
| GHS Flammability | Non-flammable | Non-flammable |
| Classification | | |

As can be seen from the results of the test work reported in Example 8, the formulation compatibility of formulation 8A and formulation 8B, in which the propellant consists of trans-1234ze and the carrier consists of trans-1233zd, fails because applicants have found that such formulations tend to cause the valve to stick at least partially in the open position, which in turns has a negative impact on the ability to reliably expell the formulation over repeated instances of use. However, the formulations are still classified as non-flammable according to GHS classification because there are less than 1% GHS classified flammable component in the formulation.

### Example 9

Example 8 is repeated except the carrier is formulated to comprise, consist essentially of, or consists of ethanol, which is a flammable component, and trans-1233zd according to the formulations indicated in Table 9 below:

TABLE 9

| Components, | Formulation | Formulation | Formulation | Formulation | Formulation |
|---|---|---|---|---|---|
| Weight % | 9A | 9B | 9C | 9D | 9E |
| Trans-1234ze | 80 | 85 | 85 | 90 | 90 |
| Trans-1233zd | 10 | 9 | 8 | 4 | 3.5 |
| Ethanol | 5 | 1.5 | 3 | 2 | 3 |
| Potato Starch | 5 | 4.5 | 4 | 4 | 3.5 |
| (TeraSorb ™) | | | | | |

Results

| | | | | | |
|---|---|---|---|---|---|
| Trans-1233zd to | 2 | 6 | 2.67 | 2 | 1.17 |
| Ethanol Ratio | | | | | |
| Internal | Passed | Passed | Passed | Passed | Passed |
| Compatibility | | | | | |
| Test | | | | | |
| GHS | Flammable | Non- | Non- | Non- | Flammable |
| Flammability | | flammable | flammable | flammable | |
| Classification | | | | | |

The results of Example 9 demonstrate the unexpected advantage which is achieved when the carrier comprises ethanol as co-carrier with trans-1233zd. In particular, applicants have discovered that a formulation with adequate compatibility is formed by Formulation 9A and Formulation 9E, but the amount of ethanol is too high to produce an non-flammable material to be expelled. Unexpectedly, Formulation 9B, Formulation 9C and Formulation 9D are classified as non-flammable and are able to passed the GHS enclosed-space test. Accoringly, applicants have discovered a critical range of these two components to be able to at once achieve each of the beneficial properties described herein.

### Example 10

The formula indicated in Table 10 below is formed by mixing the indicated components, except for the propellant, and introducing them into an aerosol spray can. The aerosol cans are sealed with the valve/actuator arrangement, and then the indicated propellant is added through the valve. The formulation spray pattern is then evaluated while the nozzle is held under ambient condition (about 73° F. and 66 relative humidity). After the formulation is subjected to the specific compatibility test (i.e. aerosol was actuated 2 times, 5 seconds each), then the formulation is aged at room temperature for a week and checked for vapor pressure and weight loss. The designated enclosed-space test, as per GHS for aerosol, is conducted to evaluate whether the formulation is considered as flammable under GHS requirements.

TABLE 10

| Components | Formulation 10 |
|---|---|
| Trans-1234ze | 80 |
| Ethanol | 15 |
| Personal Care PVP | 5 |
| Polymer (Advantage | |
| Revive ™) | |

Results

| | |
|---|---|
| Formulation Compatibility | Passed |
| Test | |
| GHS Flammability | Flammable |
| Classification | |

### Example 11

Example 10 is repeated except that trans-1233zd is used as carrier, instead of ethanol. The formulations tested and the test results are reported in Table 11 below.

TABLE 11

| | Formulation | Formulation |
|---|---|---|
| Components, Weight % | 11A | 11B |
| Trans-1234ze | 85 | 90 |
| Trans-1233zd | 10 | 5 |
| Ethanol | 0 | 0 |
| Personal Care PVP | 5 | 5 |
| Polymer (Advantage | | |
| Revive ™) | | |

Results

| | | |
|---|---|---|
| Formulation Compatibility | Failed | Failed |
| Test | | |
| GHS Flammability | Non-flammable | Non-flammable |
| Classification | | |

As can be seen from the results of the test work reported in Example 11, the formulation compatibility of formulation 11A and formulation 11B, in which the propellant consists of trans-1234ze and the carrier consists of trans-1233zd, fails because applicants have found that such formulations tend to cause the valve to stick at least partially in the open position, which in turns has a negative impact on the ability to reliably expell the formulation over repeated instances of use. However, the formulations are still classified as non-flammable according to GHS classification because there are less than 1% GHS classified flammable component in the formulation.

### Example 12

Example 11 is repeated except the carrier is formulated to comprise, consist essentially of, or consists of ethanol, which is a flammable component, and trans-1233zd according to the formulations indicated in Table 12 below:

TABLE 12

| Components, | Formulation | Formulation | Formulation | Formulation | Formulation |
|---|---|---|---|---|---|
| Weight % | 12A | 12B | 12C | 12D | 12E |
| Trans-1234ze | 80 | 85 | 85 | 90 | 90 |
| Trans-1233zd | 10 | 9 | 8 | 4 | 3.5 |
| Ethanol | 5 | 1.5 | 3 | 2 | 3 |
| Personal Care | 5 | 4.5 | 4 | 4 | 3.5 |
| PVP Polymer | | | | | |
| (Advantage | | | | | |
| Revive ™.) | | | | | |

Results

| | | | | | |
|---|---|---|---|---|---|
| Trans-1233zd to | 2 | 6 | 2.67 | 2 | 1.17 |
| Ethanol Ratio | | | | | |
| Internal | Passed | Passed | Passed | Passed | Passed |
| Compatibility | | | | | |
| Test | | | | | |
| GHS | Flammable | Non- | Non- | Non- | Flammable |
| Flammability | | flammable | flammable | flammable | |
| Classification | | | | | |

The results of Example 12 demonstrate the unexpected advantage which is achieved when the carrier comprises ethanol as co-carrier with trans-1233zd. In particular, applicants have discovered that a formulation with adequate compatibility is formed by Formulation 12A and Formulation 12E, but the amount of ethanol is too high to produce an non-flammable material to be expelled. Unexpectedly, Formulation 12B, Formulation 12C and Formulation 12D are classified as non-flammable and are able to passed the GHS enclosed-space test. Accoringly, applicants have discovered a critical range of these two components to be able to at once achieve each of the beneficial properties described herein.

### Example 13

The formula indicated in Table 13 below is formed by mixing the indicated components, except for the propellant, and introducing them into an aerosol spray can. The aerosol cans are sealed with the valve/actuator arrangement, and then the indicated propellant is added through the valve. The formulation spray pattern is then evaluated while the nozzle is held under ambient condition (about 73° F. and 66 relative humidity). After the formulation is subjected to the specific compatibility test (i.e. aerosol was actuated 2 times, 5 seconds each), then the formulation is aged at room temperature for a week and checked for vapor pressure and weight loss. The designated enclosed-space test, as per GHS for aerosol, is conducted to evaluate whether the formulation is considered as flammable under GHS requirements.

TABLE 13

| Components | Formulation 13 |
|---|---|
| Trans-1234ze | 80 |
| Ethanol | 15 |
| Personal Care Silica (CAB- | 5 |
| O-SIL M5 ™) | |

Results

| | |
|---|---|
| Formulation Compatibility | Passed |
| Test | |
| GHS Flammability | Flammable |
| Classification | |

### Example 14

Example 13 is repeated except that trans-1233zd is used as carrier, instead of ethanol. The formulations tested and the test results are reported in Table 14 below.

TABLE 14

| | Formulation | Formulation |
|---|---|---|
| Components, Weight % | 14A | 14B |
| Trans-1234ze | 85 | 90 |
| Trans-1233zd | 10 | 5 |
| Ethanol | 0 | 0 |
| Personal Care Silica (CAB- | 5 | 5 |
| O-SIL M5 ™) | | |

Results

| | | |
|---|---|---|
| Formulation Compatibility | Failed | Failed |
| Test | | |
| GHS Flammability | Non-flammable | Non-flammable |
| Classification | | |

As can be seen from the results of the test work reported in Example 14, the formulation compatibility of formulation 14A and formulation 14B, in which the propellant consists of trans-1234ze and the carrier consists of trans-1233zd, fails because applicants have found that such formulations tend to cause the valve to stick at least partially in the open position, which in turns has a negative impact on the ability to reliably expell the formulation over repeated instances of use. However, the formulations are still classified as non-flammable according to GHS classification because there are less than 1% GHS classified flammable component in the formulation.

### Example 15

Example 14 is repeated except the carrier is formulated to comprise, consist essentially of, or consists of ethanol, which is a flammable component, and trans-1233zd according to the formulations indicated in Table 15 below:

TABLE 15

| Components, | Formulation | Formulation | Formulation | Formulation | Formulation |
|---|---|---|---|---|---|
| Weight % | 15A | 15B | 15C | 15D | 15E |
| Trans-1234ze | 80 | 85 | 85 | 90 | 90 |
| Trans-1233zd | 10 | 9 | 8 | 4 | 3.5 |
| Ethanol | 5 | 1.5 | 3 | 2 | 3 |
| Personal Care | 5 | 4.5 | 4 | 4 | 3.5 |
| Silica (CAB-O- | | | | | |
| SIL M5 ™) | | | | | |

Results

| | | | | | |
|---|---|---|---|---|---|
| Trans-1233zd to | 2 | 6 | 2.67 | 2 | 1.17 |
| Ethanol Ratio | | | | | |
| Internal | Passed | Passed | Passed | Passed | Passed |
| Compatibility | | | | | |
| Test | | | | | |
| GHS | Flammable | Non- | Non- | Non- | Flammable |
| Flammability | | flammable | flammable | flammable | |
| Classification | | | | | |

The results of Example 15 demonstrate the unexpected advantage which is achieved when the carrier comprises ethanol as co-carrier with trans-1233zd. In particular, applicants have discovered that a formulation with adequate compatibility is formed by Formulation 15A and Formulation 15E, but the amount of ethanol is too high to produce an non-flammable material to be expelled. Unexpectedly, Formulation 15B, Formulation 15C and Formulation 15D are classified as non-flammable and are able to passed the GHS enclosed-space test. Accoringly, applicants have discovered a critical range of these two components to be able to at once achieve each of the beneficial properties described herein.

### Example 16

The formula indicated in Table 16 below is formed by mixing the indicated components, except for the propellant, and introducing them into an aerosol spray can. The aerosol cans are sealed with the valve/actuator arrangement, and then the indicated propellant is added through the valve. The formulation spray pattern is then evaluated while the nozzle is held under ambient condition (about 73° F. and 66 relative humidity). After the formulation is subjected to the specific compatibility test (i.e. aerosol was actuated 2 times, 5 seconds each), then the formulation is aged at room temperature for a week and checked for vapor pressure and weight loss. The designated enclosed-space test, as per GHS for aerosol, is conducted to evaluate whether the formulation is considered as flammable under GHS requirements.

TABLE 16

| Components | Formulation 16 |
|---|---|
| Trans-1234ze | 80 |
| Ethanol | 15 |
| Personal Care Kaolin Clay | 5 |

Results

| | |
|---|---|
| Formulation Compatibility | Passed |
| Test | |
| GHS Flammability | Flammable |
| Classification | |

### Example 17

Example 16 is repeated except that trans-1233zd is used as carrier, instead of ethanol. The formulations tested and the test results are reported in Table 17 below.

TABLE 17

| | Formulation | Formulation |
|---|---|---|
| Components, Weight % | 17A | 17B |
| Trans-1234ze | 85 | 90 |
| Trans-1233zd | 10 | 5 |
| Ethanol | 0 | 0 |
| Personal Care Kaolin Clay | 5 | 5 |

Results

| | | |
|---|---|---|
| Formulation Compatibility | Failed | Failed |
| Test | | |
| GHS Flammability | Non-flammable | Non-flammable |
| Classification | | |

As can be seen from the results of the test work reported in Example 17, the formulation compatibility of formulation 17A and formulation 17B, in which the propellant consists of trans-1234ze and the carrier consists of trans-1233zd, fails because applicants have found that such formulations tend to cause the valve to stick at least partially in the open position, which in turns has a negative impact on the ability to reliably expell the formulation over repeated instances of use. However, the formulations are still classified as non-flammable according to GHS classification because there are less than 1% GHS classified flammable component in the formulation.

### Example 18

Example 17 is repeated except the carrier is formulated to comprise, consist essentially of, or consists of ethanol, which is a flammable component, and trans-1233zd according to the formulations indicated in Table 18 below:

TABLE 18

| Components, | Formulation | Formulation | Formulation | Formulation | Formulation |
|---|---|---|---|---|---|
| Weight % | 18A | 18B | 18C | 18D | 18E |
| Trans-1234ze | 80 | 85 | 85 | 90 | 90 |
| Trans-1233zd | 10 | 9 | 8 | 4 | 3.5 |
| Ethanol | 5 | 1.5 | 3 | 2 | 3 |
| Personal Care | 5 | 4.5 | 4 | 4 | 3.5 |
| Kaolin Clay | | | | | |

Results

| | | | | | |
|---|---|---|---|---|---|
| Trans-1233zd to | 2 | 6 | 2.67 | 2 | 1.17 |
| Ethanol Ratio | | | | | |
| Internal | Passed | Passed | Passed | Passed | Passed |
| Compatibility | | | | | |
| Test | | | | | |
| GHS | Flammable | Non- | Non- | Non- | Flammable |
| Flammability | | flammable | flammable | flammable | |
| Classification | | | | | |

The results of Example 18 demonstrate the unexpected advantage which is achieved when the carrier comprises ethanol as co-carrier with trans-1233zd. In particular, applicants have discovered that a formulation with adequate compatibility is formed by Formulation 18A and Formulation 18E, but the amount of ethanol is too high to produce an non-flammable material to be expelled. Unexpectedly, Formulation 18B, Formulation 18C and Formulation 18D are classified as non-flammable and are able to passed the GHS enclosed-space test. Accoringly, applicants have discovered a critical range of these two components to be able to at once achieve each of the beneficial properties described herein.

1. A device for applying a hair and/or skin and/or scalp care product to a user, said device comprising:
A) a container having a spray nozzle for alternatively opening and closing the container and upon opening expelling at least a portion of contents therefrom;
B) a personal care formulation in said container comprising:
a) an active component for treating the hair and/or scalp and/or skin of the user;
b) carrier for the active component, said carrier comprising:
i) from about 4% to about 10% by weight based on the contents of the container of trans-1233zd and
ii) from about 1% to less than 5% by weight based on the contents of the container of ethanol, where in the weight ratio of said trans-1233zd:ethanol is at least about 2; and
c) at least about 70% by weight based on the contents of the container of a non-flammable propellant.

2. The device of claim 1, wherein said active component comprises a personal care oil absorbing component present in the personal care formulation in an amount of from about 3% to about 15% by weight.

3. The device of claim 2, wherein said active component comprises one or more of: personal care starch; personal care cleansing polymer; personal care silica; personal care clay; and combinations of these.

4. The device of claim 3, wherein said active component comprises a personal care starch.

5. The device of claim 4, wherein said personal care starch comprises one or more of rice starch, tapioca starch, corn starch, potato starch, aluminum starch and talc-starch, wherein the weight ratio of said trans-1233zd:ethanol is from about 2 to about 6, and wherein the weight ratio of said trans-1233zd:starch is from about 0.75:1 to about 3:1.

6. A method of applying a hair and/or skin and/or scalp care product to a user comprising:
A) providing a container having a spray nozzle for expelling at least a portion of contents therefrom;
B) including in said container a personal care formulation comprising:
a) an active personal care component;
b) carrier for the active component, said carrier comprising:
i) from about 4% to about 10% by weight based on the contents of the container of trans-1233zd and
ii) from about 1% to less than 5% by weight based on the contents of the container of ethanol, wherein the weight ratio of said trans-1233zd:ethanol is at least about 2; and
c) at least about 70% by weight based on the contents of the container of a non-flammable propellant; and
C) spraying said personal care formulation from the container and onto the area of the user to receive the personal care product.

7. The method of claim 6, wherein said active component comprises from about 2% by weight to 8% by weight based on the weight of the contents of the container of starch for absorbing oil from the hair and/or scalp and/or skin of the user.

8. The method of claim 6, wherein said personal care oil absorbing component is present in the personal care formulation in an amount of from about 3% to about 15% by weight.

9. The method of claim 7, wherein said starch comprises one or more of rice starch, tapioca starch, corn starch, potato starch, aluminum starch and talc-starch.

10. The method of claim 9, wherein the weight ratio of said trans-1233zd:ethanol is from about 2 to about 6 and wherein the weight ratio of said trans-1233zd:starch is from about 0.75:1 to about 3:1.
